(19) United States
(12) Patent Application Publication
(10) Pub. No.: US 2015/0038875 A1
(43) Pub. Date: Feb. 5, 2015

(54) DEVICE FOR COLLECTING FAECES, A METHOD FOR MANUFACTURING AND USE OF SUCH A DEVICE

(71) Applicant: FuBuSc B.V., Heiloo (NL)

(72) Inventor: Dennis Cornelis Wilhelmus Poland, Heiloo (NL)

(73) Assignee: FUBUSC B.V., Heiloo (NL)

(21) Appl. No.: 14/382,341

(22) PCT Filed: Mar. 1, 2013
(86) PCT No.: PCT/NL2013/050134

§ 371 Date: Sep. 2, 2014

(30) Foreign Application Priority Data
Mar. 1, 2012 (NL) 2008396

(51) Int. Cl. A61B 10/00

(52) U.S. Cl. CPC A61B 10/0038
USPC 600/562; 29/426.2

(57) ABSTRACT

A device for collecting feces includes a loop attachable to a toilet seat having a width dimension and a height dimension, whereby the loop has a width and a circumferential length greater than twice the width and the height of the toilet seat, such that the loop encircles the toilet seat, with a portion of the loop between covered parts of the toilet seat extending downwards, forming a collection portion to collect the feces. A method for manufacturing the device, includes: providing at least one layer of sheet material; separating the sheet of material in portions having a length dimension and a width dimension, whereby the length dimension is larger than the width direction; and separating the portion of sheet material in a longitudinal direction into several strips of sheet material having a first end and a second end. A method of using the device is also described.

### BACKGROUND OF THE INVENTION

1. Field of the Invention

The present invention relates to a device for collecting faeces, more particular human faeces. The invention further relates to a method for manufacturing a device for collecting faeces. In addition, the invention relates to a use of a device for collecting faeces.

2. Description of the Related Art

Several medical procedures require analysis of a patient's faeces sample. For example, an early detection of bowel cancer can be facilitated by faeces sample analysis. Faeces sample collection kits are handed to the patient to collect a sample.

Such a faeces sample kit must avoid cross-contamination by both toilet water and urine, or other human waste, to provide accurate information regarding the health of a patient providing a sample. However, many previously proposed faeces sample collection kits are difficult to use and cross-contamination is difficult to avoid. In addition, many faeces sample collection kits are compatible with only a limited range of toilets and so many patients will find that such kits do not accurately fit the dimensions of the rim of their toilet bowl or their toilet seats.

In addition, after the faeces sample has been collected successfully, the collection kit with the remainder of the faeces has to be disposed of The difficult and distasteful nature of the test and the disposal afterwards puts up a large barrier for patients to collect a sample of their faeces.

Accordingly, it is desirable to provide a faeces sample collection device which is easy to use, is hygienic for a user, minimizes the possibility of cross contamination with both toilet water and urine, is compatible with a range of sizes of toilet bowls and is easy to dispose of after taking the faeces sample.

A device for collecting faeces is described in US2007/0245486, the contents of which are hereby incorporated by reference in their entirety. That collecting device is designed to slidingly fit over an open ring toilet seat, forming an envelope having a rearwardly facing opening which receives the toilet seat within upper and lower portions and upper and lower sheets. A collection portion is disposed between portions of the envelope such that when the device is positioned on a toilet seat, the collection portion will extend downwardly permitting collection of human urine and faeces from a patient. The device comprises at least one removable collection portion and means for substantially closing the collection portion after the faeces has been deposited therein.

Such a collecting device designed as an envelope covers the complete toilet seat between the sheets of the envelope. The collection portion therefore stretches over the entire open section of the toilet seat, increasing the risk of contamination of the collected faeces with urine or other human waste.

In addition, the sheets are joined at the collection portion in order to have a removable collection portion, making such a device only suitable for open ring toilet seats.

It would therefore be desirable to provide an alternative container construction that alleviated at least some of the perceived inconveniences of the prior art.

### BRIEF SUMMARY OF THE INVENTION

According to the invention there is provided a device for collecting faeces, comprising a loop attachable to a toilet seat having a width dimension and a height dimension, whereby the loop has a width and a circumferential length greater than twice the width and the height of the toilet seat, such that the loop encircles the toilet seat, with a portion of the loop between the covered parts of the toilet seat extending downwards, forming a collection portion to collect the faeces.

The loop encircles the toilet seat, which seat has an upper and a lower side. The loop covers part of the upper and the lower side of the toilet seat. A loop can be used for any kind of toilet seat, being an open ring or closed ring toilet seat. In addition, the loop can be used for any shape of toilet seat, being round, elliptical, square, extended or the like. The collection portion extends downward from the lower side of the toilet seat, into the toilet bowl.

By using a loop with a circumferential length greater than twice the width and the height of the toilet seat, the loop is not taut between the covered parts of the toilet seat. Instead, the loop has a collecting portion that extends downwards into the toilet bowl. The advantage of such a downward extending collecting portion is that a distance between a person and the faeces is created, such that the faeces are not disturbed and possibly contaminated after being collected.

The distance between the upper side of the toilet seat and the downwardly extending collecting portion is created by the extra length of the loop in addition to twice the width and height of the toilet seat. This extra length should be about 20-30% of twice the width of the toilet seat, preferably 25%, to create a suitable distance between the upper side of the toilet seat and the collection portion. This distance should remain smaller than the height of a toilet bowl, such that the collecting portion will not touch the toilet water in the bowl.

The circumferential length of the loop should be about 100 cm for toilet seats with a width of about 40 cm. The circumferential length of the loop will be larger for wider toilet seats and smaller for narrower toilet seats. In practice, the circumferential length of the loop will be 90-200 cm, preferably 100-150 cm.

The width of the loop can be adjusted to only cover part of the toilet seat, such that only the faeces are collected and will not be contaminated by urine or the like. The width of the loop should not cover more than 50% of the toilet seat, preferably not more than 35-40%. For a toilet seat having a length of about 45 cm, the width of the loop should therefore be between 16-18 cm and not exceed 23 cm (about half of the length). The width of the loop will be larger for longer toilet seats and smaller for shorter toilet seats. In practice, the width of the loop will be 15-25 cm, preferably 15-20 cm.

According to an aspect of the invention, the loop is an endless loop that is slidable onto the toilet seat. It is therefore very easy to attach the loop to the toilet seat and collect the faeces sample. In addition, the loop can be used for any kind or shape of toilet seat. The loop is slid onto the toilet seat, which is then rotated downwards to rest on the rim of the toilet bowl. The collecting portion can be created by gently pushing the loop downwards, without damaging it, in case the collecting portion is not formed automatically with the downward rotation of the toilet seat. The endless loop can be constructed by connecting the free ends by means of an adhesive or other connecting elements, such as stitches through the free ends to form a sewn connection.

According to another aspect of the invention, the loop comprises a strip with two free ends, foldable around the toilet seat, whereby the two free ends of the strip connect to form a loop. In addition, it is possible to form the collecting device from a strip of material with two free ends. The strip is folded around the toilet seat, without tightening it. The free ends of the strip are connected to form a loop. The free ends can be connected to each other, but it is also possible that the free ends connect to part of the formed loop. The connection can be done by connecting means, such as velcro, hook-and-eye-fastener or an adhesive, provided on at least one of the free ends for forming a loop, or on each of the free ends for connection to part of the formed loop. The connecting means can be releasable for disposing of the collecting device.

According to a further aspect, the width of the loop covers only part of the toilet seat, such that only faeces are collected by the collecting device. By only covering part of the toilet seat, contamination of the faeces with other human waste, such as urine or other liquids of the human body, is avoided.

A further aspect is that the loop comprises a tear line for tearing the loop. After collecting the faeces and taking the sample, the collecting device has to be disposed of. By providing a tear line across at least a part of the width of the loop, the loop can be torn after the sample collection and be disposed of into the toilet bowl and flushed down the toilet.

In order to prevent the toilet or the drain pipes to get clogged, the loop comprises a water soluble and degradable material. Water soluble and degradable means that upon contact with water, the structure of the material used starts to deteriorate such that the strength decreases rapidly and the material ultimately falls apart. The dissolution and degradation rate of the material used should be comparable to that of commonly used toilet paper.

According to a further aspect, the loop comprises tissue paper. Tissue paper has a dissolution and degradation rate that is comparable to toilet paper. By using several layers of tissue paper, the desired strength can be obtained such that the faeces can be collected without falling through the device. The tissue paper is made of woodfree fibres and is creped with a ratio between 80 to 95%, preferably 90%. The weight of the material, in particular tissue paper, should be in a range of 35 to 60 grams per square meter, preferably at least 40 grams per square meter. More preferably, the weight of the material is about 50 grams per square meter. Tissue paper with a weight of about 50 grams per square meter combines good strength and good dissolution and degradation properties. In addition, the strength of paper is determined by the following factors in combination: the strength of the individual fibres of the stock, the average length of the fibre, the interfibre bonding ability of the fibre, which is enhanced by the beating and refining action, and the structure and formation of the sheet.

Resistance to rupture when subjected to various stresses is an important property in practically all grades of paper. Most papers require a certain minimum strength to withstand the treatment received by the product in use; but even where use requirements are not severe, the paper must be strong enough to permit efficient handling in manufacture. Tensile strength is the greatest longitudinal stress a piece of paper can bear without tearing apart. The stress is expressed as the force per unit width of a test specimen.

Since the weight of the paper and the width of the test specimen affect the force of rupture, a conventional method of comparing inherent paper strength is the breaking length—that is, the length of a paper strip in metres that would be just self-supporting. This value varies from about 500 metres for extremely soft, weak tissue to about 8,000 metres for strong kraft bag paper, and to about 14,000 metres for sheets of paper made under ideal laboratory conditions.

Preferably, the machine direction breaking length of the paper used for the device is between 3.75 and 4.25 km, preferably about 4.0 km and the cross direction breaking length is between 1.75 and 2.25 km, preferably about 1.9 km.

Additionally, the preferred machine direction tensile strength of at least 1.35 kN/m, preferably between 1.50 and 1.60 kN/m. The preferred cross direction tensile strength is at least 0.65 kN/m, preferably between 0.70 to 0.80 kN/m

One of the oldest and most widely used strength tests for paper and paperboard is the bursting test, or Mullen test. It is defined as the hydrostatic pressure (caused by liquids at rest) necessary to cause rupture in a circular area of a given diameter. The bursting strength of the preferred tissue paper for the device is between 80 to 90 kPa, preferably about 85 kPa.

The moister level of the tissue paper can be between 1-10%, preferably between 4-7%.

The above properties of the tissue paper make that it suffices the flushability rules for flushing materials through a toilet.

The most important optical properties of paper are brightness, colour, opacity, and gloss. The term brightness has come to mean the degree to which white or near-white papers and paperboard reflect the light of the blue end of the spectrum (i.e., their reflectance). This reflectance is measured by an instrument that illuminates paper at an average angle of incidence of 45° and a wavelength of 457μ (microns). Brightness measured in this way is found to correlate closely with subjective estimates of the relative whiteness of paper. The preferred brightness of the paper used for the device is between 75-85%, more preferred is between 76-80% of brightness, without optical brightness additives (OBA).

According to a further aspect, the collecting portion of the loop comprises a water resistant recess for collecting liquid faeces. Faeces samples can be taken for any kind of reason. Sometimes a disease can cause liquid faeces, containing water. Normally, the water in the liquid faeces starts to disintegrate the structure of the collecting device. In order to be able to collect this kind of faeces, a water resistant recess is provided in the collecting portion of the loop. The water resistant recess collects the liquid faeces, upon which the sample can be taken. Preferably, the recess comprises dissolution retardant material for decreasing the dissolution rate of the collecting device when collecting the liquid faeces. The dissolution retardant material can comprise a glue or other material that can be adhered to or impregnated into the material of the loop. Alternatively, the recess comprises a water resistant cup. The cup collects the liquid faeces, after which the sample can be taken. Preferably, the cup comprises fibrous cardboard. This material can be dissolved and degraded by water, however at a much lower rate than commonly used toilet paper, and can therefore be flushed down the toilet as well.

According to an aspect, the cup and loop form an integral collecting device. The cup can be readily attached to the loop during manufacture. In this case, the faeces, whether ‘normal’ or liquid, are always collected by the cup in the loop.

Alternatively, the cup is attachable to the loop. In this case, the person using the collecting device can choose between the loop for ‘normal’ faeces or the loop with cup for collecting liquid faeces.

The invention also relates to a method for manufacturing a device for collecting faeces, comprising:
providing at least one layer of sheet material;
separating the sheet of material in portions having a length dimension and a width dimension, whereby the length dimension is larger than the width direction;
separating the portion of sheet material in a longitudinal direction into several strips of sheet material having a first end and a second end;
providing at least the first end of the strip of material with connecting means;
connecting the first and second ends of the strip of material to form a loop.

The collecting device is manufactured from a sheet material, comprising at least one layer. The sheet material is separated by cutting, tearing, or other separating method, into portions that are relatively long and narrow. These relatively long and narrow portions are then separated in a longitudinal direction into several strips of sheet material. The obtained separate strips can be preformed as an endless loop. Therefore, at least one of the free ends of the strip is provided with connecting means, which can be an adhesive tape or glue. The free ends can then be connected to each other to form a loop. Alternatively, the free ends can be sewn together by stitching to form a sewn connection.

According to an embodiment, the method comprises before separating the sheet material into portions:
rolling the layer of sheet material to form a roll;
separating the roll into portions;
unrolling the portions;
separating the unrolled material into strips of sheet material having a length dimension and a width dimension, whereby the length dimension is larger than the width direction and having a first end and a second end.

When the sheet material is relatively large, it can be advantageous to roll the sheet into a roll, forming a relatively long and narrow cylinder. The cylinder is then separated into equal portions. The resulting smaller rolls are then unrolled and the unrolled material is separated into strips of sheet material that are relatively long and narrow.

According to a further embodiment, at least two layers of sheet material are provided, which layers are connected to each other. In order to achieve a strong collecting device for faeces, it can be advantageous to use two layers of sheet material. The layers are connected together to benefit from the additional strength. Preferably, the two layers of material are joined through embossing.

According to a further embodiment, the method comprises:
providing a tear line along the width dimension of the strip.

In order to be able to dispose of the collecting device, the loop is provided with a tear line across the width of the strip along which the loop can be torn and easily disposed of by falling into the toilet bowl and flushing the toilet.

According a further embodiment, the method comprises:
folding the strip of material;
packaging the folded strip of material individually.

The collecting device is packed individually to ensure hygienic storage of each device. In order to reduce storage space, the strip of material is folded to smaller dimensions and subsequently packed.

Alternatively, the method can comprise before folding the strip of material:
rolling the strip of material into a roll;
pressing the rolled strip of material.

The strip can be folded along folding lines, but it is also possible to first roll the strip to form a roll and then press the roll to form a flat piece. The flattened piece is then packed individually.

Preferably, the sheet material comprises a water soluble and degradable material. When the collecting device has to be disposed of after collecting the sample by flushing the toilet, the toilet bowl and/or the drain pipes will not get clogged.

If the sheet of material is a sheet of tissue paper, the material will relatively rapidly dissolve and degrade. Preferably, the weight of the material, in particular tissue paper, is in a range of 35 to 60 grams per square meter. Within this range, the tissue paper can be dissolved and degraded by water, in particular flowing water, relatively rapid. Most preferably, the weight of the material is about 50 grams per square meter. This kind of tissue paper ensures an optimal combination of strength to carry the weight of the faeces, and a good dissolution and degradation rate.

According to a further embodiment, the method comprises:
providing a portion of the strip of material with a water resistant material for collecting liquid faeces.

Normal tissue paper cannot withstand the watery substance of liquid faeces, such that the collecting device will be damaged and probably dissolved before a sample can be taken. By providing a water resistant material to a portion of the strip of material, this can be avoided. Preferably, the water resistant material comprises dissolution retardant material for decreasing the dissolution rate of the collecting device when collecting the liquid faeces.

In addition, the invention relates to a use of a device as described or manufactured as above for collecting faeces, whereby the collecting device comprises a loop attachable to a toilet seat having a width dimension and a height dimension, whereby the loop has a width and a circumferential length greater than twice the width and the height of the toilet seat, the use comprising:
sliding the loop onto a toilet seat, such that the loop encircles the toilet seat;
forming a downward extending collection portion in the loop by adjusting the loop in a downward direction;
collecting faeces in the collection portion;
taking a sample of the collected faeces;
disposing of the collecting device and the collected faeces by flushing the toilet.

According to an aspect, the loop comprises a strip with two free ends, which strip is foldable around the toilet seat, the use comprising before sliding the loop onto the toilet, connecting the first end and the second end of the strip to form a loop.

According to a further aspect, the loop is provided with at least one tear line across the width, and the use comprises before disposing of the collecting device and the collected faeces, tearing the loop along the tear line.

### BRIEF DESCRIPTION OF THE DRAWINGS

The features and advantages of the invention will be appreciated upon reference to the following drawings of a number of exemplary embodiments, in which:

FIGS. 1a-e shows an embodiment of the collection device used on a toilet seat;

FIG. 2a and b show another embodiment of the collection device comprising a water resistant collection portion;

FIG. 3 shows a schematic overview of a first stage of an embodiment of a method for manufacturing a collection device;

FIG. 4 shows a schematic overview of two embodiments of a second stage of a method for manufacturing a collection device.

### DESCRIPTION OF ILLUSTRATIVE EMBODIMENTS

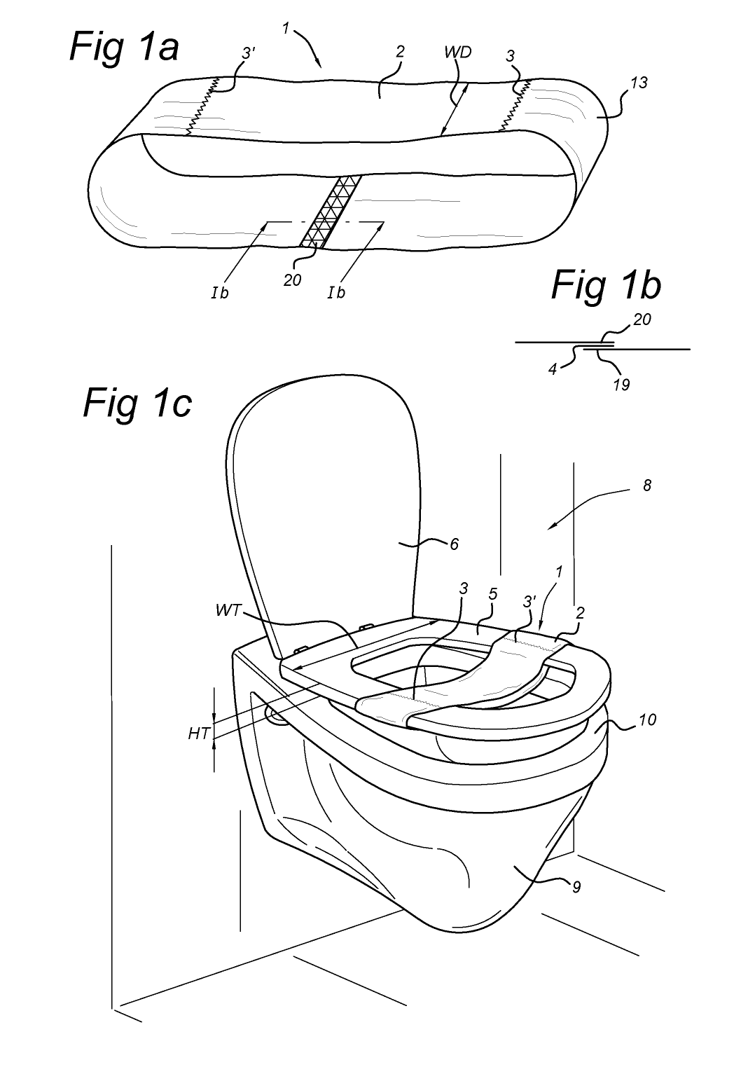

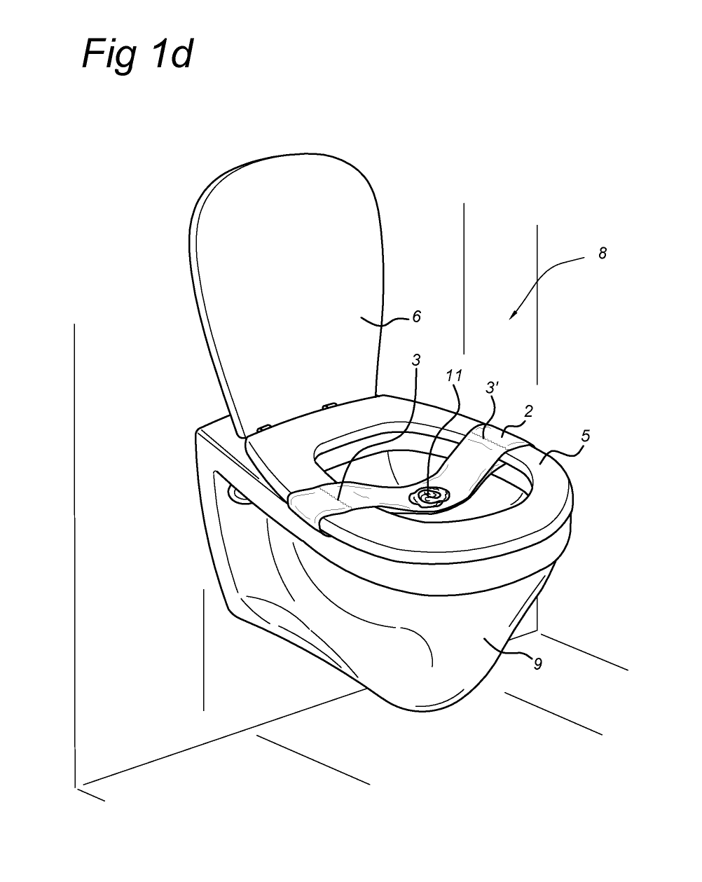

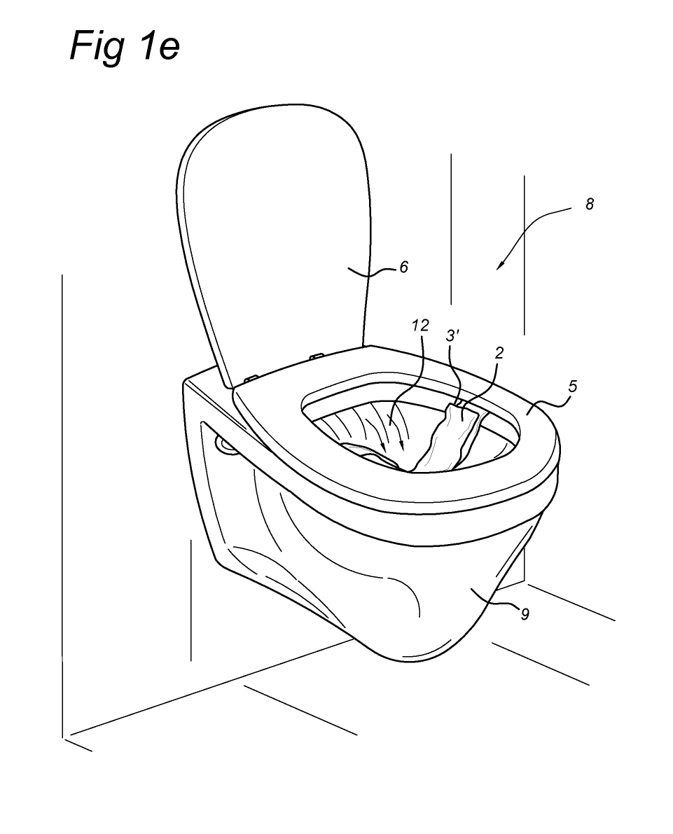

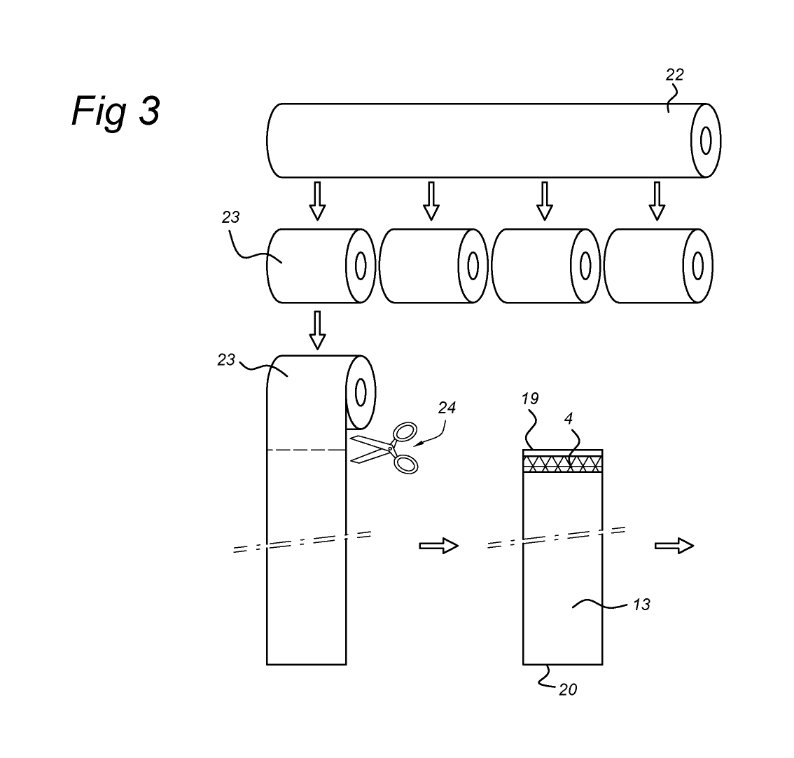

FIG. 1a shows a collecting device 1 for collecting faeces, in particular human faeces, comprising a loop 2 with a circumferential length. The embodiment shown comprises an endless loop 2, formed from a strip 13 with free ends 19 and 20 (see FIG. 3). The free ends 19 and 20 are connected by connecting means 4, such as velcro or an adhesive, to form an endless loop from the strip 13, as shown in the detail of FIG. 1b. The loop 2 is provided with at least one tear line 3, preferably two tear lines 3, 3′, across the width WD of the loop to be able to dispose of the collecting device afterwards.

FIG. 1c shows the collecting device formed as a loop 2, that is slid onto a toilet seat 5 that can be covered by a lid 6. The loop covers part of an upper and under side of the toilet seat. The toilet seat 5 is hingeably connected to a toilet bowl 9. The toilet bowl comprises a rim 10, on which the toilet seat 6 rests when using the collecting device 1. The toilet seat 6 and the collecting device 1 form a system 8.

The collecting device 1 is provided with a collection portion 7 that extends downwards from the toilet seat 5 into the toilet bowl 9. The tear lines 3, 3′ are situated at each side of the collection portion 7 of the in the collecting device 1. In addition, the tear lines coincide with the toilet seat 5 when the collecting device 1 is slid onto the toilet seat 5.

The collection portion 7 is arranged to collect faeces 11, as is shown in FIG. 1d. The width WD of the loop covers only part of the toilet seat 5. The toilet seat has a width dimension WT, a height dimension HT and a length dimension LT. Preferably, the loop width WD covers not more than 50% of the length LT of the toilet seat, such that only faeces 11 will be collected by the device 1 and not other human waste, such as urine. In this way, the faeces 11 will not be contaminated and a clean sample can be taken.

In FIG. 1e the faeces sample has been taken and the collecting device 1 can be disposed of. The material that is used to make the collecting device 1 is a water soluble and degradable material, preferably tissue paper with a weight of 50 grams per square meter. This kind of paper combines a good strength for collecting the faeces without breaking, and a good solubility and degradability in water.

To dispose the collecting device 1 with the remaining faeces 11, the loop 2 is torn along the tear lines 3, 3′. The separated parts of the loop 2 will fall, under the influence of gravity, into the toilet bowl and the toilet can be flushed such that the collecting device 1 with the remaining faeces 11 will be disposed of without much ado.

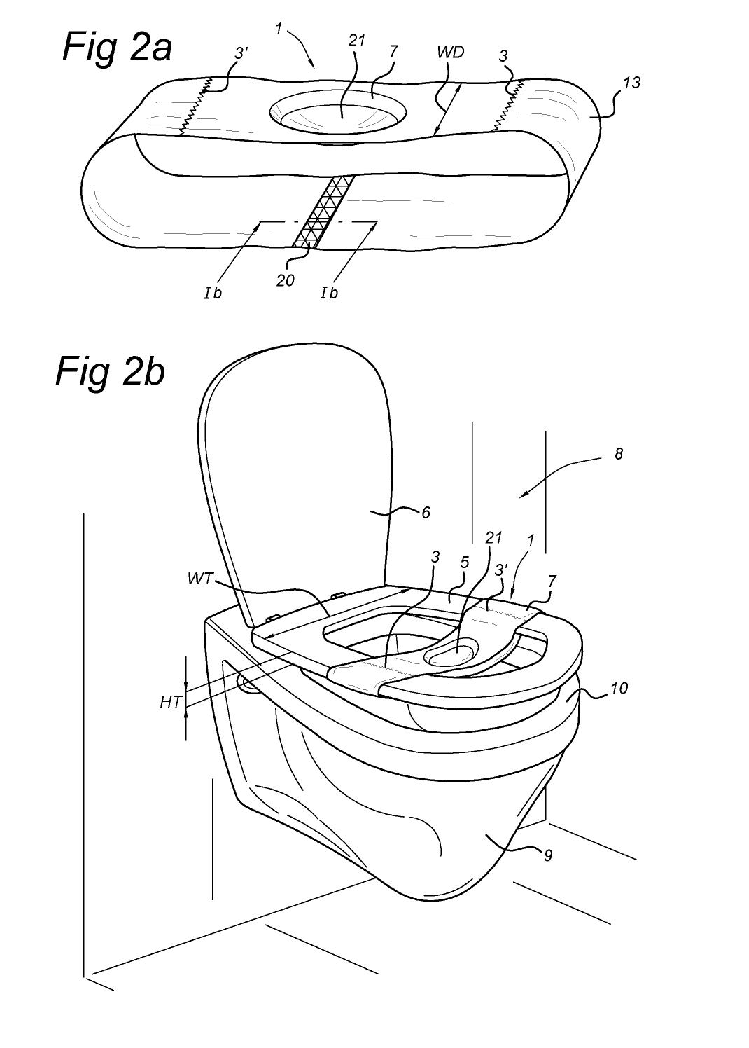

Some diseases come with the symptom of liquid faeces. When using material that is water soluble, like tissue paper, the liquid faeces cannot be collected by the collecting device, as the water in the liquid faeces start to disintegrate the collecting device upon contact. Therefore, another embodiment of the collecting device is shown in FIG. 2a and b.

FIG. 2a shows a collecting device 1 comprising a loop 2, similar to the embodiment of FIG. 1a. In addition, the collecting device 1 in FIG. 2a comprises a water resistant recess, such as a water resistant cup 21. The cup 21 and the loop 2 form an integral collecting device 1. Alternatively, the cup 21 can also be provided separately and be attachable to the loop 2 at a later stage.

FIG. 2b shows the collecting device 1 from FIG. 2a slid onto a toilet seat. The collection portion 7 that extends downwards from the toilet seat into the toilet bowl comprises a water resistant cup 21 for collecting liquid faeces. The cup 21 is made of a fibrous cardboard that has a lower dissolution and degradation rate than the tissue paper of the loop 2, but can still be flushed down the toilet without clogging the toilet bowl or the drain pipes.

FIG. 3 shows a schematic overview of a first stage of an embodiment of a method for manufacturing a collection device. First a sheet of material (not shown) comprising at least one layer is rolled to form a relatively long and narrow cylindrical roll 22. The roll 22 is separated into smaller portions 23. These portions 23 do not necessary have to be of equal size, as collecting devices with different widths can be used for different shapes of toilet seats. The smaller portions 23 are then unrolled and the unrolled material is separated by separating means into strips of sheet material 13 having a length dimension LD and a width dimension WD, whereby the length dimension LD is larger than the width direction WD, and having a first end 19 and a second end 20. An adhesive is provided on at least the first end 19. This adhesive can be a double sided adhesive tape, or glue, preferably water resistant. The first end 19 and the second end 20 can be connected either during the manufacturing process, or afterwards by the user of the collecting device 1, to form a loop 2. When the loop is formed after manufacturing and before use, the adhesive is preferably covered by a suitable material to protect the adhesive.

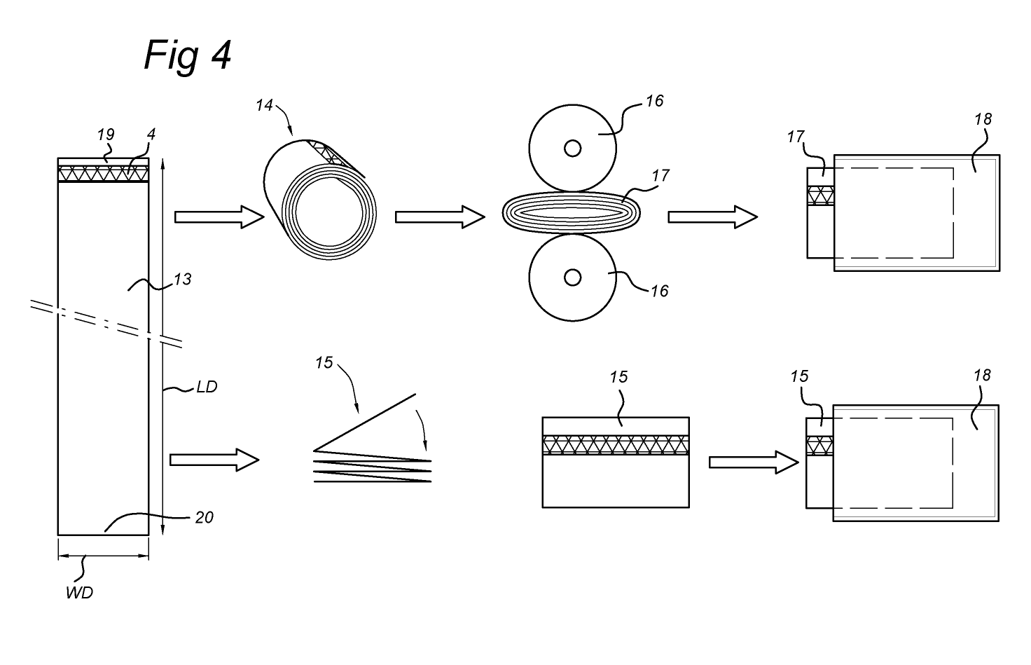

FIG. 4 shows a schematic overview of two embodiments of a second stage of a method for manufacturing a collection device. After providing the strips 13 of material, the strips 13 can be rolled to form a roll 14. The roll 14 is then pressed by pressing means 16, which can be a roller or a flat press, to form a flat object 17 with smaller dimensions than the original strip 13. By pressing the roll, relatively sharp folding lines appear in the strip 13. After pressing, the pressed rolls 17 are packed individually in a packaging 18. In this way, the storage space of the individually packed collecting devices 1 can be kept at a minimum. In addition, by individually packaging of the collecting device 1 hygienic use can be guaranteed.

An alternative way of packing the collecting devices 1 is by folding the strip 13 to a folded strip 15 along relatively sharp folding lines. The resulting folded collecting device 15 is then packed individually in packaging 18.

Thus, the invention has been described by reference to certain embodiments discussed above. It will be recognized that these embodiments are susceptible to various modifications and alternative forms well known to those of skill in the art.

Many modifications in addition to those described above may be made to the structures and techniques described herein without departing from the spirit and scope of the invention. Accordingly, although specific embodiments have been described, these are examples only and are not limiting upon the scope of the invention.

### LIST OF PARTS

1. Collecting device
2. Loop
3+3′. Tear line
4. Adhesive
5. Toilet seat
6. Lid
7. Collection portion
8. System of toilet seat and collecting device
9. Toilet bowl
10. Rim
11. Faeces
12. Toilet water
13. Strip of material
14. Rolled strip
15. Folded strip
16. Press
17. Pressed roll
18. Package
19. First free end of strip
20. Second free end of strip
21. Water resistant recess
22. Rolled sheet
23. Portioned rolled sheet
24. Separating means

1-31. (canceled)

32. Device (1) for collecting faeces (11), comprising a loop (2) attachable to a toilet seat (5) having a width dimension (WT) and a height dimension (HT), whereby the loop has a width (WD) and a circumferential length greater than twice the width and the height of the toilet seat, such that the loop encircles the toilet seat, with a portion of the loop between the covered parts of the toilet seat extending downwards, forming a collection portion (7) to collect the faeces, characterized in that the loop comprises a water soluble and degradable material.

33. Device according to claim 32, whereby the loop is an endless loop that is slidable onto the toilet seat.

34. Device according to claim 32, whereby the loop comprises a strip (13) with two free ends (19, 20), foldable around the toilet seat, whereby the two free ends of the strip connect to form a loop.

35. Device according to claim 32, whereby the width of the loop covers only part of the toilet seat, such that only faeces are collected by the collecting device.

36. Device according to claim 32, whereby the loop comprises a tear line (3, 3′) for tearing the loop.

37. Device according to claim 32, whereby the collecting portion of the loop comprises a water resistant recess (21) for collecting liquid faeces.

38. Device according to claim 37, whereby the recess comprises dissolution retardant material for decreasing the dissolution rate of the collecting device when collecting the liquid faeces.

39. Device according to claim 37, whereby the recess comprises a water resistant cup (21).

40. Method for manufacturing a device (1) for collecting faeces (11), comprising:
providing at least one layer of sheet material comprising a water soluble and degradable material;
separating the sheet of material in portions having a length dimension and a width dimension, whereby the length dimension is larger than the width direction;
separating the portion of sheet material in a longitudinal direction into several strips (13) of sheet material having a first end and a second end (19, 20);
providing at least the first end of the strip of material with connecting means (4);
connecting the first and second ends of the strip of material to form a loop (2).

41. Method according to claim 40, comprising before separating the sheet material into portions:
rolling the layer of sheet material to form a roll (22);
separating the roll into portions (23);
unrolling the portions;
separating the unrolled material into strips (13) of sheet material having a length dimension and a width dimension, whereby the length dimension is larger than the width direction and having a first end (19) and a second end (20).

42. Method according to claim 40, comprising:
providing a tear line (3, 3′) along the width dimension of the strip.

43. Method according to claim 40, comprising:
providing a portion of the strip of material with a water resistant material (21) for collecting liquid faeces.

44. Method according to claim 43, whereby the water resistant material comprises dissolution retardant material for decreasing the dissolution rate of the collecting device when collecting the liquid faeces.

45. Method of collecting faeces (11), comprising:
providing a collecting device comprising a loop (2) attachable to a toilet seat (5) having a width dimension (WT) and a height dimension (HT), whereby the loop has a width (WD) and a circumferential length greater than twice the width and the height of the toilet seat;
sliding the loop onto a toilet seat, such that the loop encircles the toilet seat;
forming a downward extending collection portion (7) in the loop by adjusting the loop in a downward direction;
collecting faeces in the collection portion;
taking a sample of the collected faeces; and
disposing of the collecting device and the collected faeces by flushing the toilet.
